(19) United States
(12) Patent Application Publication
(10) Pub. No.: US 2008/0220021 A1
(43) Pub. Date: Sep. 11, 2008

(54) Topical Botulinum Toxin Compositions for the Treatment of Hyperhidrosis

(76) Inventor: Pankaj Modi, Ancaster (CA)

Correspondence Address: HEENAN BLAIKIE LLP, P. O. BOX 185, SUITE 2600, 200 BAY STREET, SOUTH TOWER, ROYAL BANK PLAZA, TORONTO, ON, M5J 2J4, CA

(21) Appl. No.: 12/126,594

(22) Filed: May 23, 2008

Related U.S. Application Data
(63) Continuation-in-part of application No. 11/259,778, filed on Oct. 27, 2005.
Application No. 11/259,778 is a continuation-in-part of application No. 11/057,481, filed on Feb. 14, 2005.

(51) Int. Cl. A61K 39/08; A61P 43/00

(52) U.S. Cl. 424/239.1

(57) ABSTRACT

The use of a topical botulinum toxin composition is disclosed for the treatment of a disorder amenable to treatment by botulinum toxin. The disorder may comprise the symptoms associated with hyperhidrosis. The topical botulinum toxin composition comprises phospholipid micelles, one or more primary stabilizers, and one or more skin penetration enhancers.

### RELATED APPLICATIONS

This patent application is a continuation-in-part of U.S. patent application Ser. No. 11/259,778, filed Oct. 27, 2005, entitled “Stabilized Compositions for Topical Administration and Methods of Making Same”, which is a continuation-in-part of U.S. patent application Ser. No. 11/057,481, filed Feb. 14, 2005, entitled “Stabilized Protein Compositions for Topical Administration and Methods of Making Same”.

### FIELD

This patent application relates to a method for treating hyperhidrosis and, in particular, to a pharmaceutical composition for the treatment of hyperhidrosis.

### BACKGROUND

Hyperhidrosis is a condition characterized by abnormally increased and uncontrollable perspiration, in excess of that required for thermoregulation. Primary hyperhidrosis is a chronic idiopathic disorder of excessive sweating that mainly affects the axillae, hands and feet. However, any part of the body may be affected. If the hyperhidrosis is due to another medical condition, it is termed secondary hyperhidrosis.

Hyperhidrosis can cause significant psychological distress in affected patients. Patients with hyperhidrosis commonly complain of anxiety, embarrassment, stress, loss of self-esteem and confidence, and a consequent reduction in their quality of life. Also, if the patient has axillary hyperhidrosis (i.e. overactive sweat glands in the axillae), there is usually an additional problem of significantly increased body odor. Hyperhidrosis can also have detrimental physical effects. Profuse sweating can also cause skin maceration and secondary microbial infections.

Primary hyperhidrosis is a chronic condition without cure and as such, requires a safe and lasting treatment with minimal, if any, side effects.

At present, the only lasting treatments are surgical, and consist either of sympathectomy, i.e. localized destruction of the sympathetic nerve trunk which enervates the sweat glands, or the removal of the sweat glands by excision, curettage, or liposuction. However, these methods carry the general risks associated with surgery, and can also lead to compensatory sweating. Moreover, sympathectomy is of limited benefit for treatment of axillary hyperhidrosis.

Other therapies that have been shown to reduce or control sweat production include iontophoresis, topical application of aluminum chloride and administration of anticholinergic agents and beta-blockers. However, these latter treatments are typically ineffective, short acting, or not well tolerated.

Iontophoresis, or the use of an electromotive force to propel bio-active agents transdermally, has been used to treat axillary hyperhidrosis. However, iontophoresis is cumbersome and inconvenient, as it typically entails daily or weekly treatments which must be performed on an on-going basis. Iontopheresis is also uncomfortable for the patient as it involves wet sponges wrapped around metal electrodes that must be inserted under each armpit and a low-voltage current applied to the skin, producing a stinging sensation.

Antiperspirants containing aluminum-based compounds may also be used to reduce sweating in patients with hyperhidrosis. Aluminum-based complexes, e.g. aluminum chloride, aluminum chloride hexahydrate, and aluminum-zirconium compounds, react with electrolytes in excreted sweat to form a gel plug in the duct of the sweat gland. The plugs prevent the gland from excreting sweat and are removed over time by the natural sloughing of dead skin. For patients with hyperhidrosis, a much higher than normal concentration (typically 15% w/w or higher) of aluminum compound must be applied in order to be effective. Application of aluminum-based antiperspirants often must be discontinued because of skin irritation.

The use of anticholinergic agents and beta-blockers to treat hyperhidrosis may have substantial negative side effects. In certain instances, the surgical removal of sweat glands or sympathectomy may be preferable to the use of such neuroactive drugs.

While botulinum toxin is known to be useful cosmetically for smoothing out fine lines and wrinkles in the skin, injections of botulinum toxin have also been used for the treatment of hyperhidrosis. Botulinum toxin acts by blocking the release of acetylcholine from overactive cholinergic sudomotor nerve fibres. These nerves innervate the eccrine sweat glands, so by inhibition of the firing of the sudomotor nerves, excessive sweating is reduced.

However, there are several disadvantages associated with the current use of botulinum toxin. The major limitation of botulinum toxin treatment of hyperhidrosis is that the injections are painful, particularly for hand and foot injections, and require some form of anesthesia. Also, a patient must continue to obtain injections as the effect of the toxin wears off eventually. A course of treatments is often expensive. Also, there is the risk that if the injection is not done properly, there may be undesirable paralysis of surrounding muscles.

A further disadvantage associated with the current use of botulinum toxin formulations is long-term storage stability. Commercially available formulations of botulinum toxin are not stable at room temperature or even when refrigerated. For example, BOTOX Cosmetic® is supplied as a single patient use vial, and the product monograph states that once opened and reconstituted it should be stored in a refrigerator (2° to 8 C.°) and used within four hours.

Thus, there is a need for alternative methods for treating hyperhidrosis.

### SUMMARY

In one aspect, this patent application discloses a method of treating hyperhidrosis, comprising topically administering an effective amount of a botulinum toxin composition to the skin of a patient, wherein the botulinum toxin composition comprises botulinum toxin encapsulated in phospholipid micelles, one or more primary stabilizers, and one or more skin penetration enhancers.

The botulinum toxin composition may be administered at a dose of 0.01 to 20 Units of botulinum toxin per affected site per day for a period of 1 day to 8 weeks. Preferably, the botulinum toxin composition is administered at a dose of 0.1 to 10 Units of botulinum toxin per affected site per day for a duration of 1 to 2 weeks. More preferably, the botulinum toxin composition is administered at a dose of 1 to 5 Units for duration of 2 days to 10 days. Most preferably, the botulinum toxin composition is administered at a dose of 2 to 3 Units of botulinum toxin per affected site per day for a duration of 5 to 7 days.

The phospholipid micelles may comprise sphingosine and cerebroside, for example. The primary stabilizers may comprise elastin and collagen, for example. One or more of the skin penetration enhancers may be selected from the group that includes d-limonene, allantoin, fulvic acid, myrrh, hydroquinone glyquin, quillaja saponaria (QTS), and acanthophyllum squarrusom (ATS).

In another aspect, this patent application discloses the use of a botulinum toxin composition for the treatment of a disorder that is amenable to treatment by botulinum toxin, wherein the botulinum toxin composition comprises sphingosine, cerebroside, elastin, collagen and one or more skin penetration enhancers. One or more of the skin penetration enhancers may be selected from the group that includes d-limonene, allantoin, fulvic acid, myrrh, hydroquinone glyquin, quillaja saponaria (QTS), and acanthophyllum squarrusom (ATS).

In a preferred embodiment, the botulinum toxin composition comprises:
approximately 1 to 40% w/w collagen;
approximately 1 to 40% w/w elastin;
approximately 0.1 to 15% w/w sphingosine phospholipid; and
approximately 0.1 to 15% w/w cerebroside phospholipid.

The botulinum toxin composition may include additional ingredients to form a cream, lotion, gel, ointment, or spray.

### BRIEF DESCRIPTION OF THE DRAWINGS

Embodiments of the invention will be described, by way of example only, with reference to the appended drawings, wherein:

FIG. 1 illustrates an SDS polyacrylamide gel analysis of stabilized botulinum toxin;

FIGS. 2A and 2B illustrate representative Western Blot analyses of the botulinum toxin composition of the invention; and

FIGS. 3A to 3D are before and after photographs of hyperhidrosis patients treated with the botulinum toxin composition of the present invention.

### DETAILED DESCRIPTION

Methods of preparing a pharmaceutical or cosmetic composition for topical delivery of at least one active agent (such as botulinum toxin), and compositions prepared by these methods, will now be described. Thereafter, the results of a trial involving the use of a botulinum toxin composition on the symptoms of hyperhidrosis will be discussed.

The composition comprises an effective amount of an active ingredient encapsulated in phospholipid micelles, one or more primary stabilizers, and one or more skin penetration enhancers. The composition may be formulated in any form suitable for topical or transdermal administration.

### Method of Preparation of Composition—Overview

The topical composition is prepared by encapsulating an active ingredient in a phospholipid micelle. The micelle solution is then preferably combined with a base composition that includes one or more primary stabilizers, such as collagen and elastin. Briefly, the phospholipid is dissolved in a suitable solvent, such as an alcohol. For example, the phospholipid may be dissolved in ethanol or a mixture of ethanol and isopropanol. The alcohol is removed by, for example, rotary vacuum evaporation. An aqueous solution containing the active ingredient is then added. The active ingredient thus becomes encapsulated by a phospholipid micelle structure. This solution can then be combined with a base solution comprising collagen and elastin.

### Composition Component: Active Ingredient

Suitable active ingredients for inclusion in the composition include Botulinum toxin Type A, Type B, Type C, Type D, Type E, Type F, and Type G. Other active ingredients include, but are not limited to androgens, androstenediol and androisoxazole (for anabolic disorders), testosterone (hypogonadism, muscle wasting, male impotence, postmenopausal symptoms in women), dehydrotestosterdne (hypogdnadism, muscle wasting), dehydroepiandrostenone (muscle wasting, fat reduction, fitness); estrogens (postmenopausal symptoms, birth control), 17 betaestradiol, estradiol-3,17-diacetate, estradiol-3-acetate, estradiol-17-acetate, estradiol-3, 17-valerate, estradiol-3-valerate, estradiol-17-valerate, ethinyl estradiol, estrone; progesterones (prevent endometriosis, prevent endometrial cancer, control habitual abortion, suppress or synchronize estrus, promote hair growth), progesterone (preg-4-ene-3,20-dione), norethindrone, norgestrieone, norgestadienone, norgestrel, norgestimate, progestogenic acid, dihydroprogesterol, nomagesterol. The testosterone hormone may be used in any of its usual forms, such as, acetate, propionate, 17-beta-cyclopentanepropionate, enanthanate, isobutyrate, undeconate, and the like. Similarly, the estradiols may additionally be used in any of the known or newly developed forms, such as, for example, pivalate, propionate, cypionate, benzoate and other esters.

Other active ingredients may also be delivered using the composition, including: insulin, insulin like growth factors, vaccines, glucagon-like peptide (GLP), Insulin-like Growth Factor (IGF), heparin, hirugen, hirulos, huridine, mumps, measles and rubella vaccine, typhoid vaccine, hepatitis A vaccine, hepatitis B vaccine, herpes simplex virus, bacterial toxoids, cholera toxin B-subunit, influenza vaccine virus, bordetala pertussis, vaccinia virus, adenovirus, canary pox, polio vaccine virus, plasmodium falciparum, bacillus calmette geurin (BCG), klebsiella pneumoniae, HIV envelope glycoproteins, bovine somatropine, estrogens, androgens, insulin growth factors, interleukin-1, interleukin-I1 and cytokins, small molecule drugs such as NSAID, narcotics, and various other peptides, genetic material and small molecules.

Other active ingredients include various types of anti-oxidants. Some examples of free radical scavengers that may be included in the composition include, but are not limited to, Vitamins A, C and E, the minerals Zinc and Selenium, lycopene, the amino acid N-Acetyl-Cysteine and natural plant extracts of Grape Skin, Bilberry and Green Tea.

The composition can include agents that promote healing. For example, vasodilators, such as nitroglycerin and glycerin mononitrate can be encapsulated in a phospholipid micelle and then combined with collagen and/or elastin in a lotion or cream formulation and applied to the skin. Without being limited by the explanation, it is thought that the formulation of vasodilators in the composition enhances the rate of penetration as compared to administration via, for example, a skin patch. Inclusion of hydrogen peroxide and/or a perfluorocarbon may further enhance oxygenation and healing.

The composition may contain a single active ingredient or multiple active ingredients in the same composition. Various combinations of active ingredients are contemplated for inclusion in the composition.

### Composition Component Phospholipid Micelles

Various types of phospholipids can be used to form the micelles that encapsulate the active ingredient. Sphingosine is one preferred phospholipid. Sphingolipid or sphingosine-1-phosphate has been recognized as a bioactive molecule involved in the regulation of cell growth, differentiation, survival, and chemotaxis as well as angiogenesis and embryogenesis. Other species of ceramides or sphingolipids such as (N-acyl-sphingosine) and dihydroceramide (N-acyl sphinganine) may also be used.

Galactosylceramide (cerebroside), a metabolite of sphingolipids, is also a preferred phospholipid. Cerebroside is a myelin related protein that plays an important role in the regulation of cell growth, differentiation, survival, and chemotaxis. Cerebroside sulfates are important membrane constituents. Both sphingosine and cerebroside have been found to be excellent phospholipids for use in the composition. While either of these phospholipids can be used alone, combinations of sphingosine and cerebroside are particularly effective. When used in equal amounts, sphingosine and cerebroside form a micelle structure that provides a very effective vehicle for delivery of active ingredients. Other phospholipids, such as phosphatidyl choline or phosphatidyl serine are also highly effective, either alone or in combination. Other phospholipids that can form a micellar structure may be also be used.

### Composition Component: Stabilizers

Primary stabilizers help to preserve or maintain the biological structure (i.e. the three dimensional conformation) and/or the biological activity of the active ingredient. The primary stabilizers can be proteins or polysaccharides. Examples of protein stabilizers include hydroxyethyl starch (hetastarch), gelatin, collagen, elastin and combinations thereof. Collagen is preferred due to its ability to penetrate the skin without the aid of any penetration enhancers. However, the primary stabilizer can be a synthetic agent that does not induce an immune response or induce an attenuated immune response in a subject.

Secondary stabilizers may also be included in the composition. These secondary stabilizers may be used alone or in combination with primary stabilizers. Exemplary secondary stabilizers include, but are not limited to non-oxidizing amino acid derivatives (such as a tryptophan derivate, such as N-acetyl-tryptophan (“NAT”)), caprylate (i.e. sodium caprylate), a polysorbate (i.e. P80), amino acids, and divalent metal cations such as zinc. The composition can also include preservative agents such as benzyl alcohol, benzoic acid, phenol, parabens and sorbic acid or a cresol, such as an M-cresol.

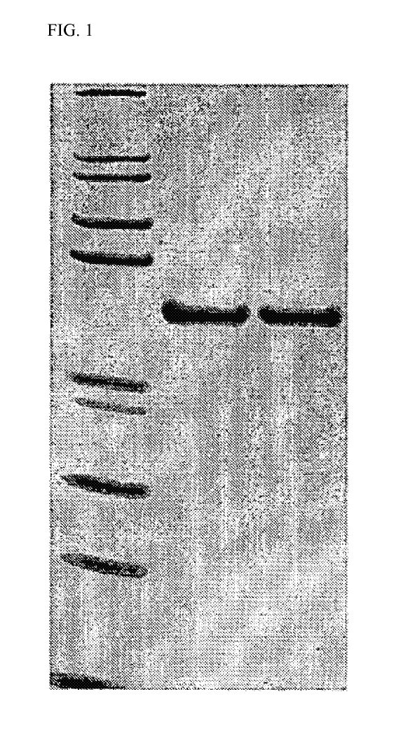

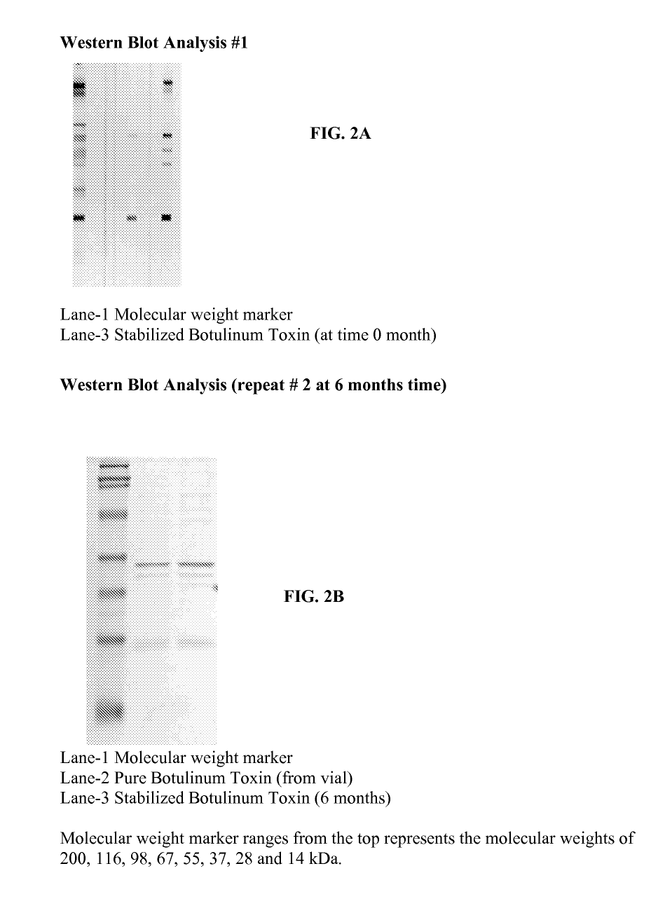

Preferably, collagen and elastin are used as primary stabilizers of botulinum toxin. A stabilized composition may be made by combining equal amounts of collagen and low molecular weight elastin in a solvent, such as saline. In a separate flask, equal amounts of sphingosine and cerebroside are dissolved in an alcohol, preferably ethanol. The alcohol is then removed by rotary vacuum evaporation. This results in a coating of sphingosine and cerebroside on the flask. A solution containing the active ingredient is added to the flask. This results in the formation of micelles of sphingosine, cerebroside and active ingredient. This micellar composition is added to the mixture of collagen and elastin and stirred. This composition stabilizes the active ingredient and enables it to be stored at room temperature for a period of months with minimal degradation, as shown in FIGS. 1 and 2.

More preferably, the composition comprises about 1 to 40% w/w collagen, 1 to 40% w/w elastin, 0.1 to 15% w/w sphingosine and 0.1 to 15% w/w cerebroside and the active ingredient, such as botulinum toxin.

### Composition Component: Surfactant

Surfactants may be used to aid in the formation of the micelles. The surfactants should be non-toxic and well-tolerated in human systems. Preferably, the surfactant used to form the micelles is phosphatidylcholine. Other amphiphilic compounds which may be used to form the micelles include other phospholipids (e.g. phosphotidylglycerol, phosphatidylethanolamine, phosphatidylinositol), and lecithin (yolk or soy derived) which itself contains phospholipids. Additional compounds which may be added to aid in formation of the micelles include other surfactants such as sodium dodecyl sulfate, lipids such as cholesterol, sodium dodecyl sulfate, and polar organic solvents such as dimethyl sulfoxide.

### Composition Component Skin Penetration Enhancer

Skin penetration enhancers promote the absorption of an active ingredient by the skin may also be included in the composition. In particular, one or more skin penetration enhancers may be used to facilitate the permeation of botulinum toxin through the patient's skin. Examples of skin penetration enhancers include, but are not limited to, alcohols, such as short chain alcohols, long chain alcohols, or polyalcohols, amines and amides, such as urea, amino acids or their esters, amides, AZONE(R)), derivatives of AZONE(R), pyrrolidones, or derivatives of pyrrolidones; terpenes and derivatives of terpenes; fatty acids and their esters; macrocyclic compounds; tensides; or sulfoxides such as, decylmethylsulfoxide. Liposomes, transfersomes, lecithin vesicles, ethosomes, water surfactants, such as anionic, cationic, and nonionic surfactants, polyols, and essential oils can also function as skin penetration enhancers.

The composition may also be used for topical administration in a format whereby the composition penetrates the skin and transdermally denervates an underlying muscle.

The composition may include d-limonene to enhance penetration of the active ingredient through the dermal layer. Limonene was found to be an effective skin penetration enhancer at 0.30%, enhancing skin permeation of botulinum toxin Type A approximately fourfold.

Quillaja saponaria (QTS) and Acanthophyllum squarrusom (ATS) total saponins are two natural skin penetration enhancers that may also be included in the composition. They demonstrate moderate activity as skin penetration enhancers.

Allantoin may also be included in the composition. Allantoin acts as a skin protectant and a mild neutral skin penetration enhancer.

Fulvic acid may also be included in the composition as a skin penetration enhancer. Fulvic acid is a low molecular weight antioxidant that enhances the body's absorption of drugs through the transdermal route.

Myrrh may also be included in the composition as a skin penetration enhancer. Myrrh is a gum resin extracted from Arabian and Somolian shrubs.

Eldopaque or hydroquinone glyquin may also be included as skin penetration enhancers.

The use of collagen in the composition, in combination with elastin and a mixture of sphingosine and cerebroside, maintains the integrity of the complex without denaturing or fragmentation or detoxification. Thus, botulinum toxin can be stabilized and the stabilized toxin can be successfully delivered transdermally to achieve similar results to those obtained by intramuscular injection of botulinum toxin. The formulation can be applied all over the face, neck, axillae and hands as opposed to a botulinum toxin injection which is targeted primarily to areas around eyes and the forehead to reduce the wrinkle.

### Additional Composition Components

The formulation may include microspheres. The formulation may be a cosmetic composition that includes water and other additives that are normally used in cosmetics. For example, it may include thickening agents, preservatives, emulsifiers, perfumes, dyes or coloring, vegetable or mineral oil, antiseptic agents, acidifying or alkalizing agents, vitamins, anti-UV agents, surfactant, solvents, pH stabilizing agents, and other active ingredients known to be effective on the skin. The composition may also be included in a make-up formulation, such as a skin foundation or a lip balm.

Additional components can be included to formulate the composition into other formats, such as a cream, lotion, spray, mask, gel, etc., that is suitable for topical administration. If formulated as a cream or a solution, the composition should contain the active ingredient in sufficiently concentrated quantities in order that the composition does not drip off the area of administation.

The composition may also be provided on a patch that is adhesively secured to the skin so that the active ingredient, such as botulinum toxin, can pass from the patch through the skin to denervate an underlying muscle.

The composition may also include aluminum salts that are known to have antiperspirant activity. Such aluminum salts include aluminum chloride, aluminum chlorohydrate, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichlorohydrate, aluminum sesquichlorohydrex polyethlene glycol complex, aluminum sesquichlorohydrex propylene glycol complex, aluminum sulfate buffered, aluminum zirconium octachlorohydrate, aluminum zirconium octachlorohydrex glycine complex, aluminum zirconium pentachlorohydrate, aluminum zirconium pentachlorohydrex glycine complex, aluminum zirconium tetrachlorohydrate, aluminum zirconium tetrachlorohydrex glycine complex, aluminum zirconium trichlorhydrate, aluminum zirconium trichlorohydrex glycine complex, and aluminum sulfate buffered with sodium aluminum lactate.

### Preparing Stabilized Botulinum Toxin Composition

A preferred method for preparing a stabilized botulinum toxin composition for topical application is described in Example 1 below.

Briefly, equal amounts of collagen and elastin were solubilized in saline. In a separate flask, equal amounts of sphingosine and cerebroside were dissolved in alcohol. The alcohol was then removed. Botulinum toxin A was dissolved in saline and then added to the flask and the flask was swirled to coat the botulinum toxin protein with a phospholipid micelle coating. This solution was then added to the solution of collagen and elastin. FIGS. 1 and 2 illustrate SDS-PAGE and Western blots of compositions of stabilized botulinum toxin at time zero compared to six months later. This method can be used to prepare compositions containing other types of botulinum toxin.

The skin penetration enhancers may be included at various stages of the fabrication process. For example, they may be added to the stabilized composition at the same time as the micellar composition is added to the collagen and elastin mixture. Preferably, the skin penetration enhancers are introduced during the formulation of the stabilized composition into a pharmaceutical or cosmetic formulation. The preparation of an exemplary formulation for topical application for hyperhidrosis is described in Example 4 below.

### Use of Composition

The composition has been demonstrated to be useful for the delivery of various agents, including botulinum toxin. The composition might be used to efficiently deliver other active ingredients to the skin, such as muscle paralysing agents, hormones, growth factors, vaccine agents, drugs, therapeutic proteins, small molecules and antiperspirant agents. The methods described herein can also be used to formulate compositions for the treatment of pain, comprising an analgesic as the active ingredient.

Compositions containing botulinum toxin and one or more skin penetration enhancers can be used to successfully treat several types of disorders associated with neurotransmitter release when applied to a person's skin. Examples of disorders amenable to treatment by the topical administration of the compositions set forth herein include, and are not limited to, wrinkles, such as brow furrows, headaches, such as migraine, headache pain, cervical dystonia, focal hand dystonia, neurogenic inflammation, hyperhidrosis, belpharospasm, strabismus, hemifacial spasm, eyelid disorder, cerebral palsy, focal spasticity, limb spasticity, tics, tremors, bruxism, anal fissure, fibromyalgia, dysphagia, lacrimation, and pain from muscle spasms. The compositions disclosed herein provide localized relief via delivery of botulinum toxin.

The composition has also been demonstrated to be useful in the treatment of the symptoms of hyperhidrosis. Details of a topical botulinum toxin composition for treating hyperhidrosis and a treatment regimen are set out below in Example 5 below.

In one treatment regimen, the topical botulinum toxin composition can be administered to hyperhidrosis patients in a dose of 0.01 Unit to 20 Units daily (as one or more applications) to an affected site (e.g. one underarm or palm each day) for a period of 1 day to 8 weeks (in other words: 0.01-20 Units per underarm or palm per day for 1 day to 8 weeks). In another treatment regimen, the botulinum toxin composition can be administered at a dose of 0.1 to 10 Units of botulinum toxin per affected site per day for a duration of 1 to 2 weeks. In another treatment regimen, the topical botulinum toxin composition can be administered to hyperhidrosis patients once daily in a dose of 1 to 5 Units of botulinum toxin per affected site (e.g. one underarm or one palm) for 2 to 10 days. In another treatment regimen, the topical botulinum toxin composition can be administered to hyperhidrosis patients once daily in a dose of 2 to 3 Units of botulinum toxin per affected site (e.g. one underarm or one palm) for 5 to 7 days.

The botulinum toxin Units referred to herein are clinically equivalent to those for the commercially marketed botulinum toxin A of the BOTOX Cosmetic® product (Allergan, Inc., California) (“Allergan Units”). As will be apparent to those skilled in the art, the dosages set out herein can be adjusted for botulinum toxins of different strengths, such as other commercially available botulinum toxins. For example, the botulinum toxin of BOTOX Cosmetic® has been reported to be four or five times more potent on a per unit basis than the botulinum toxin of DYSPORT® (Ipsen Ltd., UK). Furthermore, the botulinum toxin B of MYOBLOC® (Solstice Neurosciences Inc., California) has been reported to be 50 to 100 times less potent than the botulinum toxin A of BOTOX Cosmetic® on a per unit basis.

The topical composition may be easily self-administered by the patient in need thereof, for reducing perspiration which may be due to hyperhidrosis, and for reduction of bromhidrosis (body odour) due to perspiration.

A more complete understanding of the invention can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation. The components of the topical composition, and the topical composition itself should be pharmaceutically acceptable, i.e. those compounds, materials, compositions, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

### EXAMPLES

### Example 1

### Preparation of Botulinum Toxin Compositions

Botulinum toxin (BOTOX®) vials were reconstituted in sterile saline solution (0.9%). The vials were shaken vigorously to dissolve the botulinum toxin. The reconstituted vials were kept refrigerated and were utilized within 1 hour of reconstitution.

In a round bottom flask of 50 mL capacity, 10 mg of soluble collagen and 10 mg of elastin were combined. The mixture was solubilized in 10 mL of sterile saline solution (0.9%) with continuous stirring. In a separate 50 mL round bottom flask, 5 mg of sphingosine and 5 mg cerebroside were combined. This mixture was dissolved in 1 mL pure ethanol. The alcohol was completely removed by rotary vacuum evaporation to obtain a uniform coating of the sphingosine and cerebroside on the flask wall. To this flask 800 units of botulinum toxin solution in 6 ml of (0.9%) saline was added. The flask was swirled and then stirred continuously for 5 minutes at room temperature to uniformly coat the botulinum toxin with the sphingosine and cerebroside micelle coating. This coated and preserved micellar botulinum toxin solution was then added to the flask containing the mixture of collagen and cross-linked, low molecular weight elastin. The solution was stirred for about 5 minutes and then kept at room temperature in a brown glass vial.

### Example 2

### SDS-PAGE and Western Blot Analysis

The stability of the preserved botulinum toxin composition of Example 1 was analyzed by standard analytical techniques using SDS-PAGE and Western Blot analysis and HPLC analysis at time zero (few minutes after the preparation of the coated stabilized solution) and thereafter every month. The preparation was compared with uncoated botulinum toxin solution.

SDS-PAGE was performed in all cases under reducing conditions with a BIO-RAD® Mini-cell apparatus (Bio-Rad Laboratories, Calif.) with 10% precast tricine gels. Freshly diluted botulinum toxin (pure sample diluted with saline) was loaded in one lane and the stabilized botulinum toxin was loaded in a second lane. Both lanes were loaded with approximately 100 units of the toxin. The loading buffer, tank buffer, and the molecular weight markers were obtained from Bio-Rad Laboratories. The protein bands were visualized by the Coomassie blue staining technique. Protein concentration (density) from SDS polyacrylamide gels was measured with a BIO-RAD® densitometry system and analyzed with the standard imaging software analyzer. The comparison of the Botulinum toxin A bands, i.e. the neat botulinum toxin without stabilization and the stabilized botulinum toxin revealed no degradation in the stabilized botulinum toxin which was kept at a room temperature for 6 months. The results are shown in FIG. 1 where, starting from the left hand side, the lanes are as follows: 1. molecular weight markers (top left: 200, 116, 98, 67, 55, 37, 28 and 14 kDa); 2. botulinum toxin at time zero without stabilization; and 3 stabilized botulinum toxin composition at six months.

For Western blot analysis, proteins separated on SDS-polyacrylamide gels were transferred to nitrocellulose membranes at 25 V for 60 minutes using a BioRad protein blot module. After the complete transfer of the protein from the gel, the protein binding sites on the membranes were blocked by incubation at room temperature in 5% skim milk-Tris-buffered saline (TBS) for 60 mm. Membranes were then incubated at a room temperature with affinity-purified MAb produced against the botulinum toxin at 5 pg/ml in 5% skim milk-TBS for 4 hrs. After several washes with TBS buffer, the membranes were incubated at room temperature with affinity-purified goat anti-mouse IgG (Sigma-Aldrich, Co.) at 4 pg/ml in 5% skim milk-TBS buffer for 3 hrs. After another four to five washes with the TBS buffer, the membranes were incubated with TMB [3,3′,5,5′-tetramethylbenzidine] membrane substrate (Bio-Rad Laboratories, Calif) until color developed. This analysis revealed that after 6 months the botulinum toxin at room temperature was stable and was not degraded or denatured. The results of two exemplary Western Blots are shown in FIGS. 2A and 2B.

### Example 3

### Botulinum Toxin Cream Formulation

The stabilized botulinum toxin composition of Example 1 was formulated into a cream for topical administration as outlined below. Total Volume of the cream (400 mL):

Phase A: De-ionized Water 74.7% Tetra Sodium EDTA 0.5-0.7% Methyl Paraben 0.2% Propylene Glycol 3.0%-4.0% Glycerin 3.0%-4.0%

Phase B: Cetyl Alcohol (Ado 1 52 NE) 2.0% Cetearyl Alcohol 2.0% Glyceryl Stearate 2.0% PEG-100 Stearate 1-2% Stearic Acid (Emersol 132) 4.5% Sorbitan Palmitate 0.5-0.7% Polysorbate-85 1.0% Polysorbate 60 0.5-1% Lanolin Alcohol (Ritachol) 1.0% HoHoba Oil 0.5-1% Lanolin 1-2% Tocopheryl Acetate 0.5-1% Dimethicone 200 0.7-1.0% BHA 0.1% Propylparaben 0.1% Diazolidinyl UREA 0.2%

Phase C: Fragrance (lilac, jasmine) as needed Aloe Vera (powder) 1.5%-2.0% CoQ-10 0.5% Retinyl A 0.03-0.05% Hyaluronic Acid (pure) 1.0-1.5% Talcum Powder (Ti02) 1.0-1.5%

Phase D: d-limonene 0.7% Allantoin 0.5% Fulvic Acid 0.5% Quillaja saponaria (QTS) 0.3% Acanthophyllum squaimsom (ATS) 0.3% Myrrh Extract 0.2% Hydroquinone Glyquin 4.0%

Phase E: Stabilized Botulinum Toxin in Collagen Matrix 800 units.

Procedure: Heat Phase A and Phase B separately with agitation to 75° C. Add Phase A to Phase B and mix 30 minutes at 75° C. Cool down to 20-22° C. and then add Phase C, D and E and continue to agitate until homogenous and one phase.

### Example 4

### (a) Preparation of Botulinum Toxin A Micellar Solution

Botulinum toxin A (Botox®, Allergan) was obtained as a lyophilizate and reconstituted in sterile saline solution (0.9%). The vials were shaken to dissolve the botulinum toxin (Botox®). The reconstituted vials were kept refrigerated and were utilized, as described below, within 1 hour of reconstitution.

In a round bottom flask of 50 mL capacity, 10 mg of soluble collagen, 10 mg of elastin were weighed. The mixture was solubilized in 10 mL of sterile saline solution (0.9%). The mixture was stirred continuously.

In a separate 50 mL round bottom flask, 5 mg of phosphatidylcholine and 5 mg of cholesterol were weighed. The mixture was dissolved in approximately 1 mL of 70% ethanol. To this solution, 10 mg sodium dodecyl sulfate (SDS) and 10 mg dimethyl sulfoxide (DMSO) were added and solubilized to make a uniform homogenous solution. To this flask 1000 units of botulinum toxin (Botox®) solution in 5 ml of (0.9%) saline was added. The flask was swirled and then stirred continuously for 5 minutes at room temperature. The solution of collagen and elastin was added to this solution. The solution was stirred for about 5 minutes slowly and then kept at room temperature in a brown glass vial.

### (b) Preparation of Topical Cream

A topical base composition was prepared as noted in Table 1 below.

TABLE 1
Composition of topical base

| | Weight |
|---|---|
| Ingredient | (grams) |
| Deionized Water | qs (as needed) |
| Glycerin (humectant) | 4.00 |
| Aluminum Zirconium Complex or Alumimum | 10.0 |
| Chlorohydrate (gelling agent) | |
| Hexamethylene tetraamine (skin penetration enhancer | 8.00 |
| and coating agent, and gel forming compound) | |
| Sodium lauryl sulfate | 0.10 |
| DMSO | 0.10 |
| Alcohol Denat. | 5.00 |
| DMDM Hydantoin (preservative) | 0.20 |
| Baby powder (fragrance) | 0.10 |
| Carbopol 940 polymer or hydroxy propyl cellulose | 5.00 |
| (thickening agent) | (adjustable) |

To this base composition was added the stabilized botulinum toxin composition prepared as described above in part (a) in order to achieve a concentration of botulinum toxin type A of 2 Units per 1 mL of resultant cream. The resultant cream (the “BTX-A cream”) was stirred slowly for about 30-45 minutes, to obtain a homogeneous composition. The cream prepared in this manner was then stored at or below room temperature for future use.

### Example 5

### Hyperhidrosis Efficacy

### (a) Subjects

Subjects were included in the study if they were aged between 18 and 75 years and had idiopathic persistent bilateral primary axillary and or palmer hyperhidrosis that interfered with their daily activities. Spontaneous sweat production in each axilla of at least 50 mg, measured over 5 minutes at room temperature and at rest, was required prior to initial treatment as assessed by the gravimetric analysis or Minor's Starch-Iodine tests. Women of child-bearing potential had to have a negative urinary pregnancy test prior to each study treatment. Only subjects who were qualified and classified as healthy were included in this study.

### (b) Study of Efficacy

Initially, 40 qualified subjects were randomly assigned to receive either 5 Units of BTX-A cream (prepared in Example 4) per axilla (a total dose per day) or placebo cream containing no botulinum toxin A. The hyperhidrotic area was identified using the Minor iodine-starch test, and BTX-A cream or placebo cream was administered (by qualified nurses under the clinician supervision) evenly within the hyperhidrotic area. Subjects were followed up every week. All subjects were asked to apply the BTX-A cream or placebo cream depending on their randomization number every night before going to bed to the axilla or on their palm. Each week they were given the Minor Starch-Iodine test and visual observations were taken to see the reductions in their sweating pattern and side effects. After the 16-week follow-up visit, all subjects completing the study had an exit visit at week 24. Thereafter they were asked to visit the clinic once a month to observe their overall well-being and the disease symptoms.

### (c) Efficacy Parameters

The primary efficacy parameter was the percentage of treatment responders. This was defined as subjects who showed a reduction in axillary sweating of at least 50% of their baseline value recorded immediately prior to the most recent treatment, measured by gravimetric assessment of spontaneous axillary sweat production over 5 minutes at room temperature and at rest. The primary end point was week 2 after treatment. Other efficacy assessments included the percentage change from baseline in sweat production; the mean raw gravimetric values at each assessment; the mean duration of effect (time between treatments); the change in the size of the sweat-producing area (measured by the Minor iodine-starch test); the subjects' global assessment of treatment satisfaction (based on a 4-point scale, from +4 for complete abolishment of signs and symptoms to 0 for a worsening of signs and symptoms).

### (d) Safety Parameters

Safety was assessed by the incidence and severity of spontaneously reported adverse events (AEs) and measurement of vital signs (blood pressure, heart rate, and body temperature), skin irritation, redness, dryness, and itchiness.

### (e) Results of Study

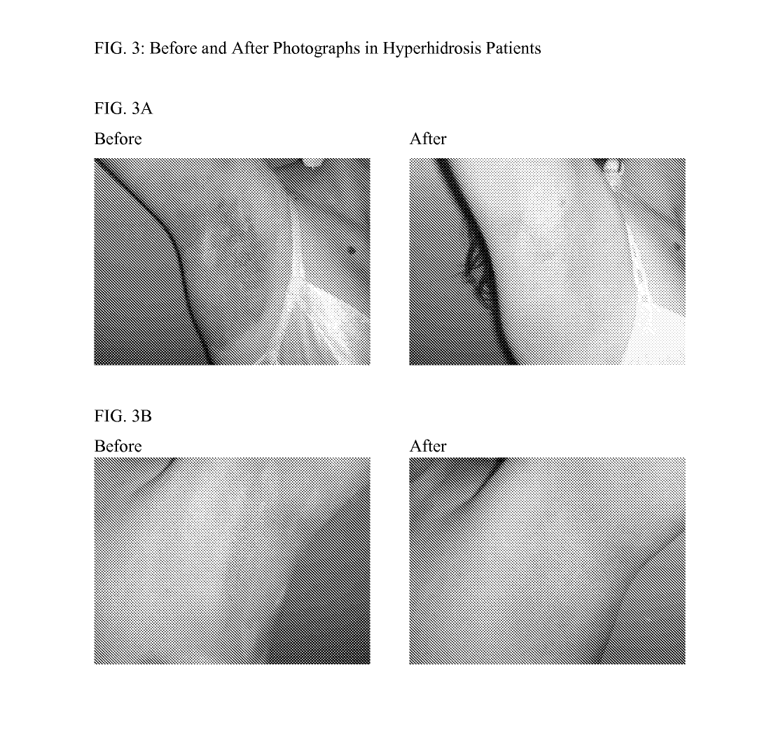

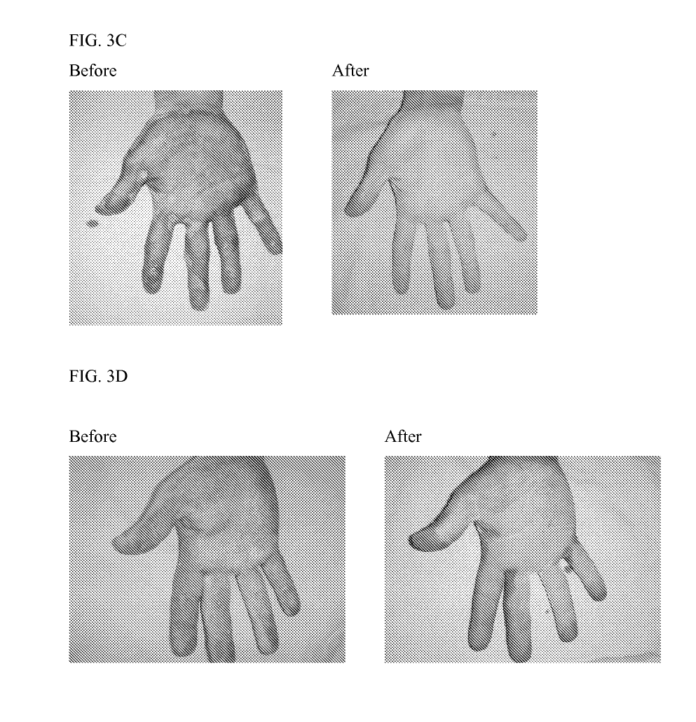

The results demonstrate that the BTX-A cream treatment is safe and efficacious for the treatment of hyperhidrosis. Exemplary results can be seen in the photographs shown in FIG. 3. As shown in FIG. 3, Minor's Starch-Iodine test was used to show symptoms of hyperhidrosis (excessive sweating) before treatment and after 4 weeks of treatment with the BTX-A cream. When the symptoms persist Minor's Starch-Iodine test stains the region black (under arm or on the palm of the hand). After 4 weeks of treatment with the BTX-A cream, Minor's Starch-Iodine test did not stain the treated region black, and similar results can be seen after 8 weeks of treatment.

An advantage of the BTX-A cream is that its use provides a simple, non-invasive, and comparatively painless means of treating the symptoms of hyperhidrosis. This is in contrast to known methods of treatment, all of which are invasive and involve discomfort or pain, such as surgical removal of sweat glands, surgical destruction of sudomotor nerves (symapthectomy), iontophoresis and intradermal injection of botulinum toxin. Moreover, the efficacy of the BTX-A cream is similar to the above-noted invasive methods of treatment.

Another advantage of the BTX-A cream is that it can be self-administered by the patient who is in need of treatment for hyperhidrosis. Patients who suffer from hyperhidrosis often complain of anxiety, embarrassment, stress, loss of self-esteem and confidence, and a consequent reduction in their quality of life. Provided with instructions for use, a patient may self-administer the composition of the invention in a private environment, and thus avoid additional anxiety.

The composition addresses many of the problems of the prior art. For example, botulinum toxin and other active ingredients can be delivered without painful injections. The composition can be safely used in areas such as the throat and neck, around the mouth, near the eye and on the hands. The composition can be formulated as a cream or lotion and can be stored at room temperature for extended periods of time with minimal loss of activity of the active ingredient. Another advantage is that the composition can be prepared without the need for blood-derived products as stabilizing agents. In addition, the composition is cost effective to prepare, and simple to use. Rather than one single large dose being delivered once to a single site, the composition can be used to administer the active ingredient at a low dose, daily, to provide an effective treatment with enhanced safety and reduced side effects.

Numerous modifications, variations and adaptations may be made to the particular embodiments of the invention described above, without departing from the scope of the invention, which is defined in the following claims.

I claim:

1. A method of treating hyperhidrosis comprising topically administering an effective amount of a botulinum toxin composition to the skin of a patient wherein the botulinum toxin composition comprises phospholipid micelles, one or more primary stabilizers and one or more skin penetration enhancers.

2. The method according to claim 1 wherein the phospholipid micelle comprises sphingosine and cerebroside.

3. The method according to claim 2 wherein the primary stabilizers are elastin and collagen.

4. The method according to claim 3 wherein the one or more skin penetration enhancers are selected from the following group: d-limonene, allantoin, fulvic acid, myrrh, hydroquinone glyquin, quillaja saponaria (QTS), and acanthophyllum squarrusom (ATS).

5. The method according to claim 3 wherein the topical administering comprises administering the botulinum toxin composition at a dose of 0.01 to 20 Allergan Units of botulinum toxin (or clinical equivalent) per affected site per day for a period of 1 day to 8 weeks.

6. The method according to claim 3 wherein the topical administering comprises administering the botulinum toxin at a dose of 0.1 to 10 Allergan Units of botulinum toxin (or clinical equivalent) per affected site per day for a duration of 1 to 2 weeks.

7. The method according to claim 3 wherein the topical administering comprises administering the botulinum toxin composition at a dose of 1 to 5 Allergan Units of botulinum toxin (or clinical equivalent) per affected site per day for a duration of 2 to 10 days.

8. The method according to claim 3 wherein the topical administering comprises administering the botulinum toxin composition at a dose of 2 to 3 Allergan Units of botulinum toxin (or clinical equivalent) per affected site per day for a duration of 5 to 7 days.

9. Use of a topical botulinum toxin composition for the treatment of hyperhidrosis, wherein the composition comprises phospholipid micelles, one or more primary stabilizers and one or more skin penetration enhancers.

10. Use of the topical botulinum toxin composition of claim 9 wherein the composition is administered at a dose of 0.01 to 20 Allergan Units of botulinum toxin (or clinical equivalent) per affected site per day for a period of 1 day to 8 weeks.

11. Use of the topical botulinum toxin composition of claim 9 wherein the composition is administered at a dose of 0.1 to 10 Allergan Units of botulinum toxin (or clinical equivalent) per affected site per day for a duration of 1 to 2 weeks.

12. Use of the topical botulinum toxin composition of claim 9 wherein the composition is administered at a dose of 1 to 5 Allergan Units of botulinum toxin (or clinical equivalent) per affected site per day for a duration of 2 to 10.

13. Use of the topical botulinum toxin composition of claim 9 wherein the composition is administered at a dose of 2 to 3 Allergan Units of botulinum toxin (or clinical equivalent) per affected site per day for a duration of 5 to 7 days.

14. A method of preparing a temperature-stabilized botulinum toxin composition, comprising:
preparing a phospholipid solution by dissolving a phospholipid in a solvent and then extracting the solvent therefrom;
preparing a micelle solution by mixing aqueous botulinum toxin with the phospholipid solution; and
mixing the coated botulinum toxin with a primary stabilizer.
